(19) United States
(12) Patent Application Publication
(10) Pub. No.: US 2026/0083716 A1
(43) Pub. Date: Mar. 26, 2026

(54) R-TRIHEXYPHENIDYL FOR TREATMENT OF MOVEMENT DISORDERS

(71) Applicant: The Children's Mercy Hospital, Kansas City, MO (US)

(72) Inventors: Rose GELINEAU-MOREL, Kansas City, MO (US); James Steven LEEDER, Kansas City, MO (US); Paul C. TOREN, Kansas City, MO (US)

(73) Assignee: The Children's Mercy Hospital, Kansas City, MO (US)

(21) Appl. No.: 19/111,178

(22) PCT Filed: Sep. 13, 2023
(86) PCT No.: PCT/US2023/074065

§ 371 Date: Mar. 12, 2025

Related U.S. Application Data
(60) Provisional application No. 63/407,219, filed on Sep. 16, 2022.

(51) Int. Cl. A61K 31/4453; A61P 25/14; C12Q 1/6883

(52) U.S. Cl. CPC A61K 31/4453; A61P 25/14; C12Q 1/6883; C12Q 2600/156

(57) ABSTRACT

Compositions and methods for selective targeting of M1 and/or M4 muscarinic receptors, compositions and methods for treating movement disorders, such as dystonia, with improved formulations of trihexyphenidyl, and in particular, compositions comprising high chiral purity R-trihexyphenidyl or enantiomerically enriched R-trihexyphenidyl.

### CROSS-REFERENCE TO RELATED APPLICATIONS

The present application claims the priority benefit of U.S. Provisional Patent Application Ser. No. 63/407,219, filed Sep. 16, 2022, entitled R-TRIHEXYPHENIDYL FOR TREATMENT OF MOVEMENT DISORDERS, incorporated by reference in its entirety herein.

### FEDERALLY SPONSORED RESEARCH OR DEVELOPMENT

This invention was made with government support under grant no. T32 HD069038 awarded by the National Institutes of Health. The government has certain rights in the invention.

### BACKGROUND OF THE INVENTION

### Field of the Invention

The present invention relates to treatment of movement disorders through selectively targeting M1 and/or M4 muscarinic receptors that are preferentially expressed in the central nervous system (CNS) using enantiomerically enriched R-trihexyphenidyl.

### Description of Related Art

Dystonia is a movement disorder characterized by sustained or intermittent muscle contractions causing abnormal, often repetitive, movements, postures, or both. Dystonic movements are typically patterned, twisting, and may be tremulous. Dystonia is often initiated or worsened by voluntary action and associated with overflow muscle activation. One of the most common causes of dystonia in childhood is neonatal brain injury resulting in cerebral palsy, which affects 3 out of every 10,000 live births. Dystonia is underrecognized but is the primary determinant of functional impairment in cerebral palsy. There is a major unmet need for symptomatic treatment options in children with dystonia, especially those with dystonia secondary to structural brain lesions or to metabolic disorders.

Trihexyphenidyl (THP) is an antispasmodic drug used to treat stiffness, tremors, spasms, and poor muscle control. It is an agent of the antimuscarinic class and is often used in management of Parkinson's disease, among other movement disorders. The racemic mixture of THP (containing both R- and S-enantiomers in a 50:50 ratio) is currently approved for patients as a generic medication (first approved in 1949), including for treatment of dystonia. However, it is well-documented to be inconsistent in its effectiveness for treating dystonia in children with cerebral palsy. In addition, the current treatment protocol is clinically ineffective as the disposition of trihexyphenidyl in the body is poorly understood and there are no standardized dosing guidelines resulting in a wide range of initial dosages, such that treatment can require significant trial and error for the clinician and patient to find effective dosages in an individual patient. Moreover, the drug is often associated with intolerable, treatment-limiting side effects, causing patients to cease use of the drug, even when it otherwise effectively ameliorates their dystonia. Given the limitations of using the current racemic formulations of THP and the lack of otherwise effective drugs for these conditions, there remains a need for improved treatment compositions and protocols for treating dystonia in cerebral palsy, Parkinson's, and other movement disorders.

### SUMMARY OF THE INVENTION

The embodiments of the present invention, thus, present clinically important alternatives for treatment of dystonia and other movement disorders relating to abnormal excitation of striatal cholinergic interneurons. In particular, a medication selectively targeting M1 and/or M4 receptors, which are preferentially expressed in the CNS, could provide improved efficacy while decreasing side effects from binding other receptor subtypes (M2, M3, and M5) that are primarily expressed peripherally (outside the CNS). This medication would selectively target the muscarinic receptors involved in movement disorders, including dystonia, Parkinson's Disease, cerebral palsy, and the like, but would not target peripheral muscarinic receptors that are responsible for many of the side effects of current medications. This would help to treat people with dystonia or Parkinson's disease, or other conditions that could be benefited by inhibiting M1 and/or M4, by providing a medication with a more targeted site of action to improve efficacy and decrease side effects.

Thus, contemplated herein are methods for treating movement disorders by administering a therapeutically effective amount of an enantiomerically enriched R-trihexyphenidyl (FIG. 1), or a pharmaceutically acceptable salt thereof to a subject in need thereof (i.e., one suffering from a movement disorder). Preferably, the administered composition comprises purified R-trihexyphenidyl and more preferably is substantially free of the S-trihexyphenidyl enantiomer. Enantiomerically enriched or purified R-trihexyphenidyl medicaments can be used as selective/specific inhibitors of M1 and M4 receptors and more effectively treat movement disorders.

Also described herein are methods of selectively targeting M1 and/or M4 muscarinic receptors in a subject suffering from a movement disorder. The methods comprise administering a therapeutically effective amount of an enantiomerically enriched R-trihexyphenidyl, or a pharmaceutically acceptable salt thereof to the subject. Preferably, the administered composition comprises purified R-trihexyphenidyl and more preferably is substantially free of the S-trihexyphenidyl enantiomer.

Also described herein are therapeutic compositions comprising (consisting essentially of or consisting of) enantiomerically enriched R-trihexyphenidyl, or a pharmaceutically acceptable salt thereof, dispersed in a pharmaceutically acceptable carrier. In one or more embodiments, the therapeutic composition can be in a pharmaceutical or unit dosage form, such as a tablet, powder, gelatin capsules, 3-D printed individualized dosage forms, etc.

Advantageously, administering R-trihexyphenidyl as a single enantiomer may be safer and more effective because R-trihexyphenidyl is a more selective/specific inhibitor of M1 and M4 receptors, and while avoiding the side effects possibly attributed to S-trihexyphenidyl's indiscriminate interactions with M1-M5 receptors expressed outside the CNS. This is supported by the results and data shown herein that R-trihexyphenidyl and S-trihexyphenidyl enantiomers have distinct inhibitory activities at the various muscarinic receptor subtypes, as well as different clearance pathways, and are metabolized by different drug metabolizing enzymes, such as cytochromes P450 (CYPs). Collectively, the embodiments described herein have important pharmacogenomic applications for individualization of drug therapy.

Also described herein are improved treatment methods for treating and managing movement disorders by utilizing factors unique to individual patients to facilitate precision treatment. For example, embodiments described herein comprise obtaining or identifying a subject's cytochrome P450 genotype (diplotype) or genotype-predicted phenotype for drug metabolism (and drug-drug interactions) to inform treatment and dosing decisions. For example, subjects having reduced CYP2D6 or CYP3A4/CYP3A5 activity due to genetic variation (poor metabolizers) or drug-drug interactions are candidates where levels of S-trihexyphenidyl are likely to accumulate leading to a higher incidence of side effects. Such subjects are candidates for which the enantiomerically enriched R-trihexyphenidyl formulation is recommended. The methods comprise the obtaining the genotype-predicted CYP450 metabolic phenotype of the individual, and administering a therapeutically effective amount of an enantiomerically enriched R-trihexyphenidyl, or a pharmaceutically acceptable salt thereof to a subject who is a CYP2D6 or CYP3A4/CYP3A5 poor metabolizer. The disclosed embodiments also relate to methods of initiating R-trihexyphenidyl treatment in a patient who is a CYP2D6 or CYP3A4/CYP3A5 poor metabolizer.

Similarly, subjects identified as fast (or ultrarapid) CYP2C19 metabolizers are candidates where levels of R-trihexyphenidyl are likely to be rapidly depleted and for whom higher initial dosages could be tolerated and even required, especially when treating with the enantiomerically enriched R-trihexyphenidyl formulation. If the subject is also a normal or fast CYP2D6 or CYP3A4/CYP3A5 metabolizer, the subject can also likely tolerate higher dosages of the racemic mixture without suffering from the side effects attributed to the S-enantiomer. The methods comprise the obtaining the genotype-predicted CYP450 metabolic phenotype of the individual, and administering a therapeutically effective amount of an enantiomerically enriched R-trihexyphenidyl or racemic mixture, or a pharmaceutically acceptable salt thereof to a subject who is a CYP2C19 fast metabolizer.

Similarly, the methods can be used for treating movement disorders in a subject who is a CYP2C19 poor metabolizer. The methods comprise the obtaining the genotype-predicted CYP450 metabolic phenotype of the individual, and administering a therapeutically effective amount of enantiomerically enriched R-trihexyphenidyl or a pharmaceutically acceptable salt thereof to the subject wherein the subject is a CYP2C19 poor metabolizer, wherein said therapeutically effect amount is a low dose of said R-trihexyphenidyl as compared to the standard clinically recommended dosage. For example, the effective dose in such an individual could be half the standard recommended dosage, preferably wherein the low dose is from 3 mg per day to 15 mg per day.

Methods disclosed herein can also be used to treat movement disorders in a subject who is a poor CYP2D6 or CYP3A4/CYP3A5 metabolizer to minimize side effects. The methods comprise the obtaining the genotype-predicted CYP450 metabolic phenotype of the individual, and decreasing an initial dose of a racemic mixture of trihexyphenidyl or a pharmaceutically acceptable salt to less than 1 mg per day and administering to the subject wherein the subject is a CYP2D6 or CYP3A4/CYP3A5 poor metabolizer, or increasing an initial dose of a racemic mixture of trihexyphenidyl or a pharmaceutically acceptable salt to more than 6 mg per day and administering to the subject wherein the subject is a CYP2D6 or CYP3A4/CYP3A5 normal or ultrarapid metabolizer, wherein the initial dose is based upon an initial dose recommended in the clinical guidelines for trihexyphenidyl.

The present disclosure also concerns methods for administering an initial dose of a M1 and/or M4 muscarinic receptor inhibitor to a subject in need thereof, where the initial dose is based upon an initial dose recommended in the clinical guidelines for the inhibitor, and wherein the inhibitor is selected from the group consisting of a racemic mixture of trihexyphenidyl, enantiomerically enriched R-trihexyphenidyl, and pharmaceutically acceptable salts thereof. The methods generally comprise obtaining the subject's genotype for a panel of cytochrome P450 enzymes comprising at least CYP2D6 and/or CYP2C19 alleles, wherein the subject is assigned a metabolic phenotype selected from poor metabolizer, intermediate metabolizer, or ultrarapid metabolizer for each enzyme based upon the number of functional alleles for each cytochrome P450 gene. The method comprises administering to the patient an initial dose of the inhibitor, wherein the initial dose is: (a) an initial dose that is half the initial dose recommended in the clinical guidelines if the metabolic phenotype is one or more of CYP2D6 poor metabolizer, CYP2D6 intermediate metabolizer, or CYP2C19 poor metabolizer; or (b) an initial dose that is the same or higher than the initial dose recommended in the clinical guidelines if the metabolic phenotype is one or more of CYP2D6 ultrarapid metabolizer or CYP2C19 ultrarapid metabolizer.

Also described herein are methods of reducing side effects from an initial dose of a M1 and/or M4 muscarinic receptor inhibitor in a subject in need thereof, where the initial dose is based upon an initial dose recommended in the clinical guidelines for the inhibitor, and wherein the inhibitor is a racemic mixture of trihexyphenidyl or the pharmaceutically acceptable salts thereof. The methods comprise obtaining the subject's genotype for a panel of cytochrome P450 CYP2D6 alleles, wherein the subject is assigned a metabolic phenotype selected from poor metabolizer, intermediate metabolizer, or ultrarapid metabolizer for CYP2D6 based upon the number of functional alleles for CYP2D6. The methods comprise administering to the patient an initial dose of the inhibitor, wherein the initial dose is: (a) an initial dose that is half the initial dose recommended in the clinical guidelines if the metabolic phenotype is one or more of CYP2D6 poor metabolizer or CYP2D6 intermediate metabolizer; or (b) an initial dose that is the same or higher than the initial dose recommended in the clinical guidelines if the metabolic phenotype is of a CYP2D6 ultrarapid metabolizer.

Also described herein are medicaments for selectively targeting M1 and/or M4 muscarinic receptors or treating dystonia in a subject in need thereof. The medicaments generally comprise a therapeutic composition comprising enantiomerically enriched R-trihexyphenidyl or a pharmaceutically acceptable salt thereof according to any one of the embodiments disclosed herein, or a pharmaceutical dosage form to any one of the embodiments disclosed herein.

### BRIEF DESCRIPTION OF THE DRAWINGS

FIG. 1 shows the molecular structure of trihexyphenidyl and shows the location of a) the chiral center (the chiral carbon) and the resulting b) R-enantiomer and c) S-enantiomer, which have identical chemical structures, but differing in three-dimensional space.

FIG. 2 presents data identifying the CYP metabolic pathways involved in the biotransformation (metabolism) of R-trihexyphenidyl and S-trihexyphenidyl.

FIG. 3 shows the results of in vitro incubations in which R-trihexyphenidyl (1,000 ng/ml) and S-trihexyphenidyl (1,000 ng/ml) are incubated with heterologously expressed CYP2C19, CYP2D6 and CYP3A4, and metabolites produced showing that R-THP-M1 is almost exclusively formed by CYP2C19 whereas S-THP-M2 is primarily formed by CYP2D6.

FIG. 4A-B, FIG. 4C-D, and FIG. 4E-F exemplify the interindividual variability in CYP2C19- and CYP2D6-dependent metabolite plasma concentrations in three patients having different CYP2C19 and CYP2D6 genotypes, when dosed of racemic trihexyphenidyl prescribed, and concurrent administration of an inhibitor of CYP2C19 activity. FIG. 4A-B shows graphs of the plasma concentration-time profiles of (A) R-trihexyphenidyl and (B) S-trihexyphenidyl for Patient 1 with an intermediate metabolizer (IM) genotype for CYP2C19 (CYP2C19*1/*2), a normal metabolizer (NM) genotype for CYP2D6 (CYP2D6*1/*2) and concurrently receiving an inhibitor of CYP2C19, esomeprazole 40 mg per day.

FIG. 4C-D shows graphs of the plasma concentration-time profiles of (C) R-trihexyphenidyl and (D) S-trihexyphenidyl for Patient 2, with an intermediate metabolizer (IM) genotype for CYP2C19 (CYP2C19*2/*17), an intermediate metabolizer (IM) genotype for CYP2D6 (CYP2D6*1/*4) and concurrently receiving an inhibitor of CYP2C19, omeprazole 20 mg per day.

FIG. 4E-F shows graphs of the plasma concentration-time profiles of (E) R-trihexyphenidyl and (F) S-trihexyphenidyl for Patient 3, with a normal metabolizer (NM) genotype for CYP2C19 (CYP2C19*1/*1), an poor metabolizer (PM) genotype for CYP2D6 (CYP2D6*4/*4) and NOT concurrently receiving an inhibitor of CYP2C19.

FIG. 5A-B illustrates the consequences of interindividual variability in CYP2C19- and CYP2D6-dependent trihexyphenidyl metabolism on the plasma concentrations of racemic trihexyphenidyl, R-trihexyphenidyl and S-trihexyphenidyl for Patient 1 from FIG. 4A-B following the (A) prescribed dose of 0.05 mg/kg racemic trihexyphenidyl and (B) normalized to a dose of 0.1 mg/kg.

FIG. 5C-D illustrates the consequences of interindividual variability in CYP2C19- and CYP2D6-dependent trihexyphenidyl metabolism on the plasma concentrations of racemic trihexyphenidyl, R-trihexyphenidyl and S-trihexyphenidyl for Patient 2 from FIG. 4C-D following the (C) prescribed dose of 0.025 mg/kg racemic trihexyphenidyl and (D) normalized to a dose of 0.1 mg/kg.

FIG. 5E-F illustrates the consequences of interindividual variability in CYP2C19- and CYP2D6-dependent trihexyphenidyl metabolism on the plasma concentrations of racemic trihexyphenidyl, R-trihexyphenidyl and S-trihexyphenidyl for Patient 3 from FIG. 4E-F following the (E) prescribed dose of 0.13 mg/kg racemic trihexyphenidyl and (F) normalized to a dose of 0.1 mg/kg (FIG. 5F).

### DETAILED DESCRIPTION

The present invention is concerned with methods for treating movement disorders with improved formulations of trihexyphenidyl, and in particular, compositions comprising high chiral purity R-trihexyphenidyl or enantiomerically enriched R-trihexyphenidyl. Thus, therapeutic compositions are described herein which comprise (consist essentially of or consist of) high chiral purity R-trihexyphenidyl or enantiomerically enriched R-trihexyphenidyl, or a pharmaceutically acceptable salt thereof, dispersed in a pharmaceutically acceptable carrier. As used herein, “high chiral purity” or “enantiomerically enriched” means that the compound is predominantly of the stated enantiomer, preferably at least 75% of the compound present is the R-enantiomer, more preferably at least 85%, even more preferably at least 95%, even more preferably at least 99% (as compared to conventional racemic mixtures of trihexyphenidyl which typically contain a 50:50 mixture of R- and S-enantiomers). Thus, the compound has preferably been purified to remove substantially all of the S-enantiomer from the racemic mixture.

Pharmaceutically acceptable salts include hydrochloride, hydrobromide, acetate, benzoate, carbonate, mesylate, bitartrate, and the like. References herein to therapeutic dosages for the enantiomer are intended to encompass the salt forms, unless otherwise indicated.

Various embodiments of the invention are therefore directed to multi-component systems, pharmaceutical compositions and methods including high chiral purity R-trihexyphenidyl or enantiomerically enriched R-trihexyphenidyl in different dosages and chiral purities than could be achieved using the traditional racemic mixture without eliciting adverse effects. For therapeutic uses, the high chiral purity R-trihexyphenidyl or enantiomerically enriched R-trihexyphenidyl is administered as part of a composition comprising a therapeutically effective amount of high chiral purity R-trihexyphenidyl or enantiomerically enriched R-trihexyphenidyl dispersed in a pharmaceutically-acceptable carrier.

The term carrier is used herein to refer to diluents, excipients, vehicles, and the like, in which the R-enantiomer may be suspended or dispersed for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with the R-enantiomer or any of the other components of the composition in which it is contained. A pharmaceutically-acceptable carrier would be selected to minimize any degradation of the R-enantiomer or other agents and to minimize any adverse side effects in the subject. In general, trihexyphenidyl is provided in a tablet form or as a liquid elixir or oral solution. Pharmaceutically-acceptable ingredients include those acceptable for veterinary use as well as human pharmaceutical use and will depend on the route of administration. Other ingredients may be included in the composition, including preservatives, buffering agents, salts, and other pharmaceutically-acceptable ingredients. For example, R-enantiomer compositions suitable for oral administration include binders or bulk excipients for pressed tablet or gel-cap forms such as colloidal silicon dioxide, dibasic calcium phosphate, lactose monohydrate, magnesium stearate, microcrystalline cellulose, sodium starch glycolate, starches, and the like, while liquid elixirs or oral suspensions include alcohol 5%, citric acid, parabens, sodium chloride, sugar alcohol (e.g., sorbitol), flavoring agents, and the like.

The composition can comprise a therapeutically effective amount of the R-enantiomer dispersed in the carrier. As used herein, a “therapeutically effective” amount refers to the amount that will elicit the biological or medical response of a tissue, system, or subject that is being sought by a researcher or clinician, and in particular elicit some desired amelioration of dystonia or other movement disorder, such as by selectively blocking M1 and/or M4 muscarinic receptors. Thus, the R-enantiomer is preferably provided in an amount sufficient to selectively bind these receptors. One of skill in the art recognizes that an amount may be considered therapeutically “effective” even if the condition is not totally eradicated or stopped, but it or its symptoms and/or effects are improved or alleviated partially in the subject, such as reduction in the number, frequency, or severity of uncontrolled movements, spasms, or other indicators of dystonia or improvement in measurements in gross or fine motor function tasks, or recognized scales for assessing dystonia (e.g., Burke-Fahn-Marsden Dystonia Scale, Barry-Albright Dystonia Scale, Melbourne Assessment of Unilateral Upper Limb Function, etc.). It is also contemplated that improved enantiomerically enriched R-trihexyphenidyl may be used as part of a multi-faceted treatment plan.

It is also contemplated that patients may be able to achieve therapeutically effective amounts of the enantiomer with lower initial dosages as compared to the previous racemic mixtures. In general, therapeutically effective amounts may be half of the dosage necessary for the racemic mixture. It is also contemplated that subjects may be able to tolerate higher dosages that would achieve even greater therapeutic efficacy, since the adverse side effects normally caused by the S-enantiomer are avoided with the new treatment modality. In one or more embodiments, potential dosages of R-trihexyphenidyl could include a range from 3 mg per day to 30 mg per day or higher. That is, without wishing to be bound by theory, it is proposed that the current effective dosing recommendation of 6 mg to 60 mg per day for the racemic mixture could be cut in half when only using the R-enantiomer. Conversely, however, it is also proposed that when the treatment-limiting side effects of S-trihexyphenidyl are removed, subjects may in fact be able to tolerate even higher dosages of the enantiomerically enriched R-trihexyphenidyl (i.e., greater than 60 mg per day) with corresponding greater therapeutic efficacy (again, without the side effects typically encountered when using the racemic mixture at the same dosages).

The methods comprise administering a therapeutically effective amount of R-trihexyphenidyl or a pharmaceutically acceptable salt thereof to a subject in need thereof (i.e., one suffering from a movement disorder). Preferably, the administered composition comprises purified R-trihexyphenidyl and more preferably is substantially free of the S-trihexyphenidyl enantiomer. As used herein, “substantially free” means that the composition (racemic mixture) has been purified to remove all or substantially all of the S-enantiomers of trihexyphenidyl, and in particular the composition comprises less than 1% w/w of S-enantiomers, preferably less than 0.5% w/w, and even more preferably less than 0.1% w/w. That is, at least 99% w/w of the trihexyphenidyl present in the composition is R-trihexyphenidyl, preferably at least 99.5% w/w, and more preferably 99.9% w/w. The composition may be administered once daily, twice daily, or three times daily depending on the dosage form to achieve the daily dosage totals described herein. That is, the “effective” dose or “initial” dose refers to the totally daily dosage, which may be broken up into one, two, or three individual administrations in a day to reach the total recommended dose for that day. Extended-release dosage forms may require less frequent dosing. As noted, trihexyphenidyl is typically orally administered as a tablet or liquid elixir or oral suspension.

Advantageously, R-trihexyphenidyl is a selective/specific inhibitor of M1 and M4 receptor with activity as much as 525-fold greater than that of S-trihexyphenidyl, which also indiscriminately interacts with all five receptors (M1-M5) at comparable inhibition potency. Thus, R-trihexyphenidyl is a much more potent antimuscarinic, and improved therapeutic benefits can be obtained by using the high chiral purity R-trihexyphenidyl or enantiomerically enriched R-trihexyphenidyl, while avoiding the side effects attributable to the S-enantiomer. Moreover, the stereoselective metabolism of trihexyphenidyl means that clinicians can use pharmacogenetic testing and precision medicine to identify subjects who may be intolerant of conventional racemic mixtures of trihexyphenidyl.

Metabolic phenotyping can be obtained using any standard approach, including phenotyping based upon test probe substances, such as dextromethorphan (DXM). More commonly, however, pharmacogenetic testing from patient fluid samples (blood, serum, urine, saliva, etc.) can be used to assign a predicted metabolizer phenotype or status according to established guidelines, such as those published by the Clinical Pharmacogenetic Implementation Consortium (CPIC). Currently there are genetic assays which predict metabolic phenotype based on the presence or absence of genetic variants for various CYP450 enzymes, which result in altered metabolic clearance of a given medication for that individual. The results of the genetic assays are translated into expected metabolic phenotypes based on an understanding of the functional consequences of the particular pharmacogenetic variant, or “allele” on enzyme activity. This translational process is facilitated using publicly accessible resources. The Pharmacogene Variation (PharmVar) Consortium evaluates, catalogues, and curates reports available on pharmvar.org on allelic variation of genes impacting drug metabolism, disposition, and response and provide a unifying designation system (nomenclature) for use by the global pharmacogenetic/genomic community, including academia, the medical community, industry and regulatory agencies. The standardized nomenclature (core allele definitions) developed by PharmVar is utilized by the Pharmacogenomic Knowledge Base (PharmGKB) and the Clinical Pharmacogenetic Implementation Consortium (CPIC). PharmGKB is an NIH-funded resource that provides information about how human genetic variation affects the response to medications, and collects, curates and disseminates knowledge about clinically actionable gene-drug associations and genotype-phenotype relationships. CPIC creates, curates, and posts freely available, peer-reviewed, evidence-based, updatable, and detailed gene/drug clinical practice guidelines that follow standardized formats, including systematic grading of evidence and clinical recommendations, based upon standardized terminology, and peer-review. CPIC guidelines are posted on cpicpgx.org, published in a leading clinical pharmacology journal, indexed in PubMed endorsed by academic societies and referenced by ClinGen and PharmGKB. Coordination of activities by PharmVar, PharmGKB and CPIC ensure consistency across evaluation of new allele data submitted to PharmVar for evaluation and curation as well as evaluation of the evidence assessing the association between pharmacogene variation and drug clearance/systemic drug exposure and the resulting clinical consequences (i.e., related to drug efficacy or risk of side effects) for clinical application of pharmacogene variation data in the form of guidelines for translating genetic laboratory test results into actionable prescribing decisions for affected drugs.

Genetic variation, or polymorphisms in these enzymes can influence enzyme activity ranging from complete loss of catalytic activity in poor metabolizers to increased enzyme activity associated with fast clearance in ultra-rapid metabolizers. Generally speaking, possession of a fully functional allele on each chromosome results in assignment of “normal metabolizer” status, and the presence of non-functional variant on each chromosome results in assignment of “poor metabolizer” status. Intermediate metabolizer status generally involves diplotypes that are combinations of non-functional alleles in combination with reduced activity or partial function alleles. Ultra-rapid metabolizer status can be assigned when two or more functional copies of the gene are present on the same chromosome (e.g., duplication or multiplication events) or in the presence of alleles associated with increased activity, depending on the pharmacogene involved. The assignment of predicted function to a particular allele is specific to the gene of interest. For example, the CYP2D6*2 allele is associated with fully functional enzyme activity, whereas the CYP2C19*2 allele is associated with a complete loss of activity. Information regarding variants and the associated phenotypes for each CYP450 enzyme is continually updated and available on the PharmVar site. Other major sources of PGx guidance include clinical practice guidelines from medical organizations, the Pharmacogenomics Knowledge Base (PharmGKB), and the US Food and Drug Administration's (FDA's) drug labeling and Table of Pharmacogenetic Associations.

Once the isoforms are identified, they can be converted into an activity score which reflects the relative activity of the enzyme activity in a particular patient (see, e.g., Gaedigk, A., Simon, S., Pearce, R., Bradford, L., Kennedy, M. and Leeder, J. (2008), The CYP2D6 Activity Score: Translating Genotype Information into a Qualitative Measure of Phenotype. Clinical Pharmacology & Therapeutics, 83:234-242. doi:10.1038/sj.clpt.6100406). Activity scores and associated genotype-predicted phenotypes are continually reviewed by PharmVar, PharmGKB, and CPIC.

The genetic predicted metabolic phenotypes are categorical and labeled relative to an average individual being labeled a normal metabolizer. The common nomenclature refers to these functional phenotypes as “poor metabolizers” at one extreme and “ultrarapid metabolizers” at the other extreme. A poor metabolizer is typically an individual in which both copies of the gene (both alleles) have low activity or are nonfunctional. An ultrarapid metabolizer is typically an individual carrying at least one allele with multiplications of normal function alleles (and thus increased overall function), even though the other allele may have decreased function but is still functional and usually present in multiple copies of the decreased function allele. A “normal” metabolizer is typically an individual carrying two normal function alleles or one normal function allele and a multiplication of a decreased function allele. An “intermediate” metabolizer falls between a poor and normal metabolizer, and is typically an individual carrying one normal function allele or a duplication of a decreased function allele and one nonfunctional allele, or two decreased function alleles, or one decreased function allele and a multiplication of a decreased function allele giving rise to at least some activity level overall (above the poor metabolizer, but still less than the normal metabolizer).

Based on published methods, the “activity score” of the enzyme can be calculated based upon the individual activity value for each allele and used to assign a predicted individual functional phenotype (Gaedigk et al., 2008). In general, an overall activity score of 0 (or less than 0.5) indicates a poor metabolizer, and an activity score of 2.5 and above indicates an ultrarapid metabolizer. A normal metabolizer has an activity score of 1.5-2.0, while an activity score between 0.5 and 1 indicates an intermediate metabolizer. Information on activity scores can be found for individual enzymes on the PharmVar site, for example, pharmvar.org/gene/CYP2D6 or pharmvar.org/gene/CYP2C19. Again, CYP isoforms can be measured by a variety of commercially available kits. Further, beyond the genotype, drug-drug interactions must also be taken into consideration to account for any other xenobiotics taken by the subject which may act as CYP450 inhibitors.

Thus, in one or more embodiments, clinicians can first identify subjects in need of treatment who have a predicted functional phenotype of being a poor CYP2D6 or CYP3A4/CYP3A5 metabolizer who are ideal candidates for precision treatment. The clinician can obtain the CYP450 metabolic phenotype of the individual (or preferably at least the CYP2D6 or CYP3A4/CYP3A5 phenotype), using any suitable approach for metabolic phenotyping (including genotype-predicted phenotypes and drug-induced phenotypes). For example, pharmacogenomic testing or metabolic phenotyping can be conducted according to known protocols using test probe substances to assess the metabolites produced by the individual, or standard genotyping. Again, a variety of CYP450 assays are available and new approaches are being routinely developed. Pharmacogenomic testing for CYP450 enzyme activity are also routinely available, including interpretation of the results and assignment of the functional phenotype.

When a subject is identified as having impaired CYP2D6 or CYP3A4/CYP3A5 enzyme activity, that subject is labeled as a CYP2D6 or CYP3A4/CYP3A5 poor metabolizer. In this case, using precision or personalized medicine, the clinician is now informed that the subject may have a higher risk of adverse side effects when being treated with the conventional racemic mixture of trihexyphenidyl (due to accumulation of S-trihexyphenidyl). In such as case, the clinician can adjust the initial dosage of the racemic trihexyphenidyl based on the metabolic phenotype of the subject to minimize side effects. In contrast, a subject who is identified as a “normal” metabolizer may begin with a higher initial dosage of the racemic trihexyphenidyl and may be at less risk for adverse side effects. Nevertheless, the dose being determined via a dose titration consistent with typical clinical care as understood by one of ordinary skill in the art. In one or more embodiments, methods described herein comprise administering a therapeutically effective amount of an enantiomerically enriched R-trihexyphenidyl, or a pharmaceutically acceptable salt thereof to a subject who is a CYP2D6 or CYP3A4/CYP3A5 poor metabolizer. The disclosed embodiments also relate to methods of initiating enantiomerically enriched R-trihexyphenidyl treatment in a patient who is a CYP2D6 or CYP3A4/CYP3A5 poor metabolizer.

Similarly, in prescribing the conventional racemic mixture of trihexyphenidyl or a formulation enantiomerically enriched R-trihexyphenidyl for the treatment of movement disorders, clinicians can utilize the CYP2C19 metabolic phenotype of the individual (predicted from CYP2C19 genotype). When a patient is identified as having impaired CYP2C19 enzyme activity, that patient is assigned CYP2C19 poor metabolizer status and may be managed effectively with a lower dose of either the conventional racemic mixture of trihexyphenidyl or a formulation highly enriched in R-trihexyphenidyl. Similarly, a patient with CYP2C19 genotypes associated with increased activity, such as CYP2C19*17/*17, are assigned ultrarapid metabolizer phenotypes and may require higher than usual doses to attain therapeutic concentrations of R-trihexyphenidyl in the body. It will be appreciated that a particular advantage of using the enantiomerically enriched R-trihexyphenidyl is that by substantially removing S-trihexyphenidyl from the formulation, concerns related to the additional genetic variation from CYP2D6 isoforms is reduced or eliminated. Thus, using precision or individualized medicine, the clinician is now better informed of factors unique to the individual that may influence therapeutic benefit and risk of toxicity associated with different treatment protocols.

As such, embodiments described herein can be used to inform clinician treatment protocols in a more precise and individualized manner to improve patient outcomes. Those skilled in the art can develop the appropriate treatment plan based upon the age and condition of the patient, the patient's tolerance to side effects, as well as the severity of the condition.

In some embodiments, the high chiral purity R-trihexyphenidyl or enantiomerically enriched R-trihexyphenidyl or compositions can be provided in unit dosage form in a suitable container. The term “unit dosage form” refers to a physically discrete unit suitable as a unitary dosage for human or animal use. Each unit dosage form may contain a predetermined amount of the high chiral purity R-trihexyphenidyl or enantiomerically enriched R-trihexyphenidyl (and/or other active agents) in the carrier calculated to produce a desired effect. In one or more embodiments, the high chiral purity R-trihexyphenidyl or enantiomerically enriched R-trihexyphenidyl can be provided separate from the carrier (e.g., in its own vial, ampule, sachet, or other suitable container) for on-site mixing (e.g., at the pharmacy or at home) before administration to a subject. In one or more embodiments, the high chiral purity R-trihexyphenidyl or enantiomerically enriched R-trihexyphenidyl is provided in discrete tablets, pills, gel caps, 3-D printed individualized dosage forms, and the like.

A kit comprising the high chiral purity R-trihexyphenidyl or enantiomerically enriched R-trihexyphenidyl is also disclosed herein. The kit further comprises instructions for administering the high chiral purity R-trihexyphenidyl or enantiomerically enriched R-trihexyphenidyl to a subject. The high chiral purity R-trihexyphenidyl or enantiomerically enriched R-trihexyphenidyl can be provided as part of a dosage unit, already dispersed in a pharmaceutically-acceptable carrier, or provided separately from the carrier. The kit can further comprise instructions for preparing the high chiral purity R-trihexyphenidyl or enantiomerically enriched R-trihexyphenidyl for administration to a subject, including for example, instructions for dispersing the high chiral purity R-trihexyphenidyl or enantiomerically enriched R-trihexyphenidyl in a suitable vehicle to create the elixir or oral suspension.

It will be appreciated that therapeutic methods described herein are applicable to humans as well as for veterinary use for any suitable animal, including, without limitation, dogs, cats, and other companion animals, as well as, rodents, primates, horses, cattle, pigs, etc. The methods can be also applied for clinical research and/or study.

The embodiments described herein are suitable for treating subjects where selective inhibition of M1 and/or M4 muscarinic receptors produces a beneficial effect in the subject. For example, the embodiments described herein are suitable for treating subjects suffering from a variety of movement disorders including dystonia, or dystonia that is secondary to another condition such as Parkinson's, cerebral palsy, Angelman Syndrome, or other genetically-mediated movement disorder, and the like.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

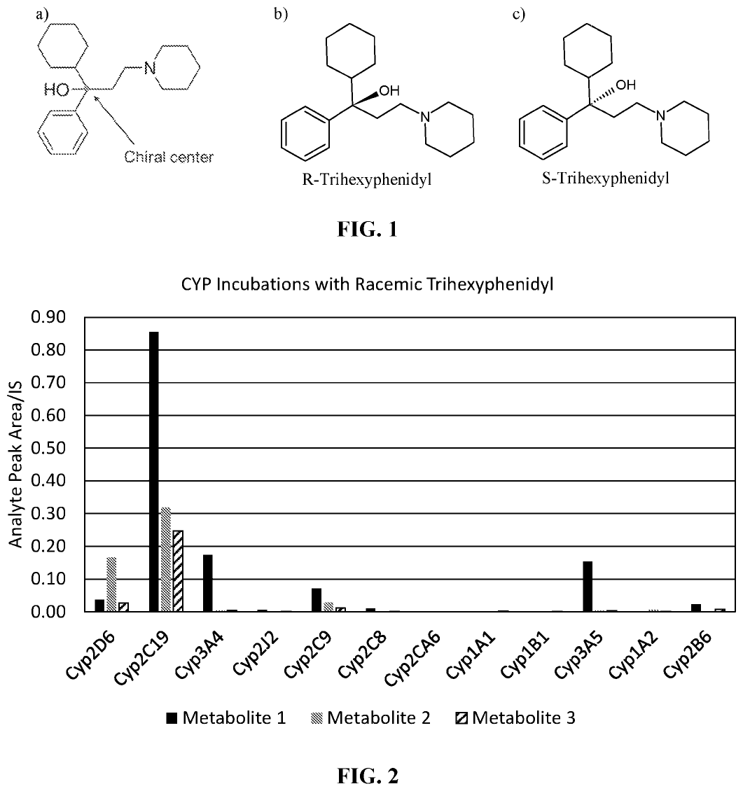

“Enantiomers” refers to asymmetric molecules that can exist in two different isomeric forms which have different configurations in space. Other terms used to designate or refer to enantiomers include “stereoisomers” (because of the different arrangement or stereochemistry around the chiral center; although all enantiomers are stereoisomers, not all stereoisomers are enantiomers). Molecules which exist in two enantiomeric forms are chiral, which means that they can be regarded as occurring in “left” and “right” handed forms. The most common cause of chirality in organic molecules is the presence of a tetrahedral carbon bonded to four different substituents or groups. Such a carbon is referred to as a chiral center, as shown in FIG. 1. Enantiomers have the same empirical chemical formula, and are generally thought of as being chemically identical in their reactions, their physical properties, and their spectroscopic properties. However, there is a growing appreciation for different pharmacokinetic profiles of enantiomers or isomers of the same chemical compound. The designations “R” and “S” are used in accordance with their customary meaning to denote the absolute configuration of the molecule about its chiral center.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Investigation of the Biotransformation of Trihexyphenidyl and Application to Dystonic Cerebral Palsy

Objective: Trihexyphenidyl (THP) is an anticholinergic medication that is commonly used for the treatment of dystonia and other movement disorders in children with cerebral palsy or with certain genetic conditions, such as Angelman Syndrome. Dystonia pathophysiology includes an increased number of striatal cholinergic interneurons as well as abnormal excitation of striatal cholinergic interneurons. Thus, anticholinergic medications, such as THP are potential treatments, and rodent models demonstrate a normalization of striatal activity after exposure to THP. For example, research in mouse models has shown that Trihexyphenidyl acts at M4 muscarinic receptors in the striatum and normalizes dopamine release associated with dystonia. Yet in clinical practice patients treated with THP demonstrate inconsistent efficacy as well as adverse effects (nausea, constipation, and urinary retention) which may be due to variations in the dose-exposure relationship. Patients often require dose reduction or stopping treatment due to adverse events. These mixed results have led to modern recommendations concluding that there is insufficient evidence regarding effectiveness of trihexyphenidyl for people with Cerebral Palsy.

In Angelman Syndrome, trihexyphenidyl can be used to treat dystonia, myoclonus, and other movement disorders. Rodent models of Angelman Syndrome demonstrate disordered dopamine release, with increased dopamine in the nucleus accumbens and decreased dopamine in the striatum, leading to movement disorders and behavioral changes. Muscarinic receptor modulation with an M4 selective anti-muscarinic agent (like trihexyphenidyl) could have the benefit of increasing dopamine release in the striatum, while muscarinic receptor antagonism of M1 and M5 receptors in the nucleus accumbens causes decreased dopamine release. As trihexyphenidyl is a selective M1, M4 muscarinic antagonist, it could potentially help with both the deficiency and excess of dopamine in patients with Angelman syndrome, making it a promising therapeutic option for treatment of movement disorders and behavioral changes.

Many drugs are metabolized in the liver by one or more (and usually a combination) of the cytochrome P450 (CYP) family of enzymes. For example, CYP2D6 is a key xenobiotic-metabolizing enzyme involved in the clearance of many drugs. Genetic polymorphisms in CYP2D6, or other enzymes such as CYP3A4/CYP3A5 or CYP2C19, contribute to the large interindividual variability in drug metabolism and hence, clearance of medications from the body. Likewise, how fast or slow someone metabolizes and/or clears a medication from the body impacts how that medication functions in their body-both in terms of the extent of therapeutic efficacy and side effects.

Most patients can be characterized as having “normal” enzyme function for various CYP450 enzymes, and readily metabolize certain drugs. However, some patients have little or no enzyme function for certain enzymes, and can be considered or assigned the status as “poor metabolizers” for those enzymes-i.e., have a substantially reduced ability to metabolize certain drugs that are metabolized via those particular enzymes. Therefore, the pharmacogenomic profile of an individual along with information about which CYP450 metabolic pathway is followed by a given medication can help predict dosage effects. The consequence of reduced enzyme activity due to pharmacogenetic variability or drug-drug inhibition is accumulation of affected medications and an increased risk of concentration-dependent side effects. Conversely, pharmacogenetic variation resulting in increased activity, such as duplication/multiplication of functional CYP2D6 variants or CYP2C19*17/*17 genotypes may result in enhanced drug clearance from the body leading to inadequate concentrations in the body and therapeutic failure. In addition, some drugs or foods act as inhibitors for CYP2D6, CYP3A4/CYP3A5, or CYP2C19 enzyme function, and therefore can inhibit or reduce the metabolism of these drugs if concomitantly administered or consumed within the time-frame that drug. The considerations can be true for other enzymes in the CYP450 family.

For example, in the context of the present disclosure, as shown herein, a patient who is a poor CYP2D6 metabolizer is predicted to have a reduced ability to metabolize and clear S-trihexyphenidyl (the enantiomer responsible for side effects) and thus have a higher likelihood of side effects when treated using the racemic mixture. In this case, the individual is a potential candidate to recommend for treatment with the enantiomerically enriched R-trihexyphenidyl. Similarly, in the context of the present disclosure, as shown herein, a patient who is a rapid CYP2C19 metabolizer is predicted to have a fast clearance of R-trihexyphenidyl (the enantiomer responsible for therapeutic effects) and thus may benefit from a higher initial dosage to ensure sufficient (therapeutically effective) plasma levels of R-trihexyphenidyl (for binding to the M1 and/or M4 receptors). And depending on whether the clinician is treating with the racemic mixture or enantiomerically-enriched R-trihexyphenidyl CYP2D6 metabolizer status, the CYP2D6 status of the individual may also be considered (if S-trihexyphenidyl metabolism is a concern).

Treatment protocols for racemic mixtures of THP currently recommend starting at a low dosage of 0.5-1 mg per day and increasing gradually by 1 mg every 3-5 days, until benefit or adverse effects. The usual effective dosage is highly variable ranging from 6 to 60 mg/day, but some patients require higher dosages. In short, very little is known about the biotransformation of THP that could inform individualized dosing strategies to provide an optimal systemic exposure for each patient. The biotransformation of THP was investigated through in vivo and in vitro analysis to determine whether new protocols could improve medication response while decreasing side effects of this otherwise promising drug.

Methods: THP and deuterated THP (D11-THP, (racemic “11D-trihexyphenidyl” in which all 11 available hydrogens on the cyclohexane ring and “5D-trihexphenidyl” in which all five available hydrogens on the phenyl ring have been deuterated) were incubated with recombinant CYP2D6, CYP2C19, and CYP3A4/CYP3A5 enzymes, and human liver microsomes. Resulting metabolites were identified and quantified using mass spectrometer. Urine samples from patients taking THP were also obtained and metabolites identified and quantified using mass spectrometer. Initial Results: Incubation of THP with recombinant CYP450 enzymes revealed a predominant CYP2D6 metabolism at lower concentrations of THP (0.2 ug/ml), which transitioned to CYP3A4 predominant metabolism at higher (supra-physiologic) concentrations (6 ug/ml). Incubation of D11-THP confirmed hydroxylation of the cyclohexane ring by both CYP2D6 and CYP3A4 yielding two, potentially three, different metabolites (two major, one minor), with the predominate metabolite as MW238. Patient urine samples revealed mixed CYP2D6 and CYP3A4 metabolite formation, with predominant CYP3A4 metabolite present in the urine of all patients, confirming the in vitro results.

Further Results: Subsequent analysis using a larger panel of CYP450 enzymes provided more insight. Racemic trihexyphenidyl (20 ng/ml) was incubated with a panel of heterologously expressed human CYPs (XenoTech LLC), and metabolites with a molecular weight equivalent to the mass of trihexyphenidyl plus an additional 16 Da, consistent with formation of hydroxylated metabolites, were separated by liquid chromatography using a chiral column (Restek Raptor® Biphenyl, 1.8 um, 100×2.1 mm) that is not able to resolve the individual enantiomers and the abundance of each metabolite determined by tandem mass spectroscopy (LC/MS/MS). Because the structures of the metabolites (location on the cyclohexane ring) remain to be established, these metabolites are designated as “Metabolite 1”, “Metabolite 2” and “Metabolite 3” and the amount of metabolite formed as assessed by integrating the area of the eluted peak in arbitrary area units. Under these experimental conditions, incubation of racemic THP with a larger panel of 12 recombinant CYPs revealed two predominant metabolites and one minor metabolite formed by 3 key CYPs: CYP2C19, CYP2D6, and CYP3A4, with minor contributions from CYP3A5 and CYP2C9 (FIG. 2). Contributions of the 3 key CYPs to metabolite formation in vitro was 51% CYP2C19, 43% CYP2D6, and 6% CYP3A4. Incubation of D11-THP with human liver microsomes confirmed that both metabolites are formed by hydroxylation of the cyclohexane ring. The same metabolites were present in patient urine and plasma samples, with predominance of metabolite 1

Conclusion: Biotransformation of THP occurs by hydroxylation of the cyclohexane ring, primarily through a combination CYP2C19, CYP2D6 and CYP3A4 metabolism, with metabolites confirmed to be present in the plasma and urine of all patients taking THP.

### Example 2

### Further Analysis of Enantiomers

Further analysis of THP has identified differences in the enantiomers of THP. The R-enantiomer has up to 525-fold greater binding affinities than the S-enantiomer, and has selective affinity for human M1 and M4 muscarinic receptors, whereas the S-enantiomer has equal affinities for all receptor subtypes. Further, our data reveals stereoselective metabolism of THP.

We performed in vitro incubations in which R-trihexyphenidyl (1,000 ng/ml) and S-trihexyphenidyl (1,000 ng/ml) were incubated with heterologously expressed CYP2C19, CYP2D6 and CYP3A4, and metabolites produced in the incubations separated as described for FIG. 2 but using a chiral column (Supelco Astec CHIROBIOTIC® V 25 cm×4.6 mm, 5 um) to separate R-trihexyphenidyl and S-trihexyphenidyl and the metabolites formed from each enantiomer. Under the chromatography conditions employed, the CYP-generated hydroxylated metabolites eluted from the column in two pairs or peaks. One pair had elution times of 10.8 and 11.2 minutes and the second pair had retention times of 12.0 and 12.9 minutes. The earlier eluting pair of metabolites are referred to as M1, with the 10.8-minute peak being formed from R-trihexyphenidyl (R-THP-M1) and the metabolite eluting at 11.2 minutes being formed from S-trihexyphenidyl (S-THP-M1). Similarly, the 12.0-minute peak is referred to as R-THP-M2 and the 12.9-minute peak as S-THP-M2.

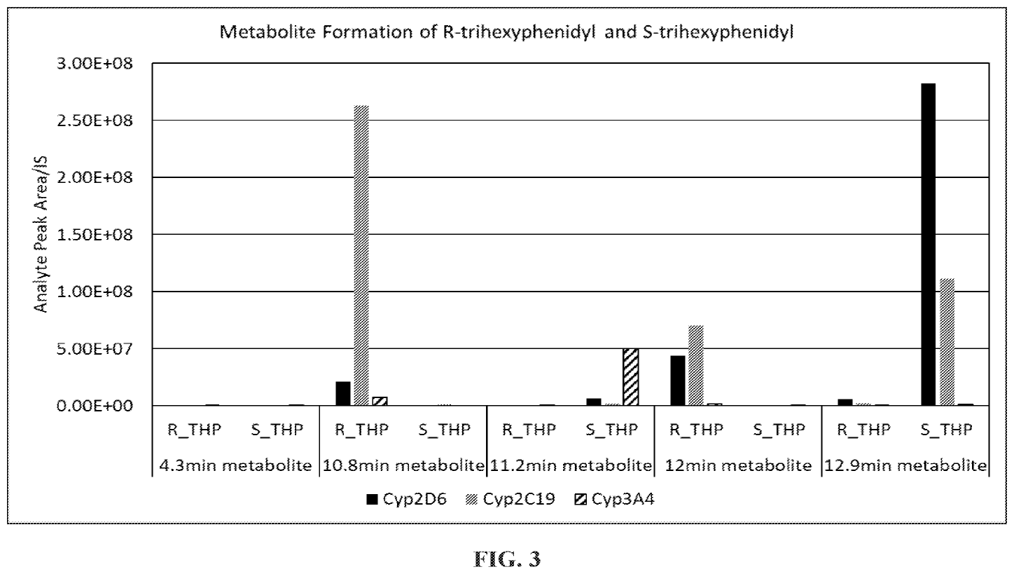

The data presented indicate that R-THP-M1 and S-THP-M2 are the most abundant metabolites formed under these experimental conditions, and R-THP-M1 is almost exclusively formed by CYP2C19 whereas S-THP-M2 is primarily formed by CYP2D6 (FIG. 3). Thus, in a patient who is a poor CYP2D6 metabolizer, S-trihexyphenidyl accumulates to higher concentrations, potentially resulting in an increased risk of side effects, while a patient who is an ultrarapid CYP2C19 metabolizer may have lower levels of R-THP, resulting in decreased effectiveness of the drug. From this new understanding, it is proposed that development of a formulation enriched in, or containing exclusively, R-trihexyphenidyl reduces or eliminates potential toxicity associated with S-trihexyphenidyl and CYP2D6 genetic variation.

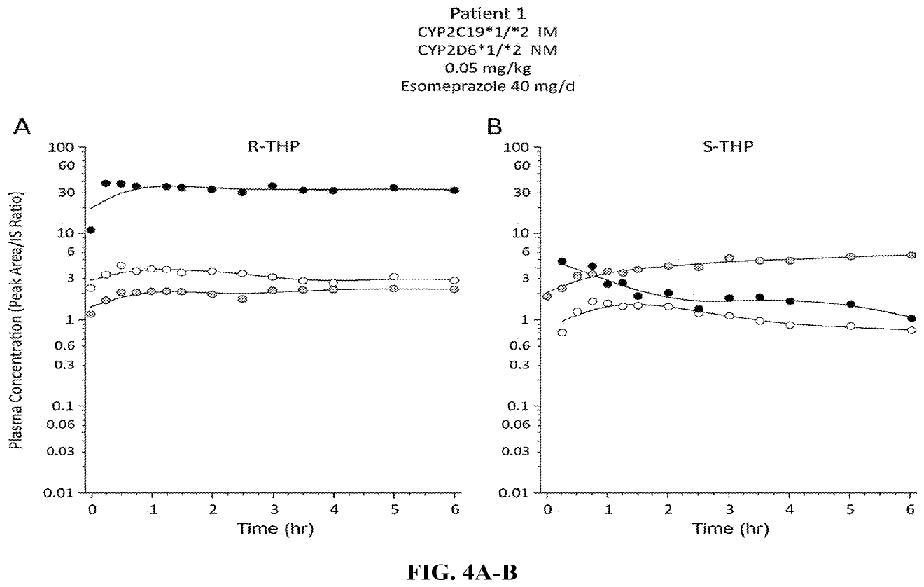

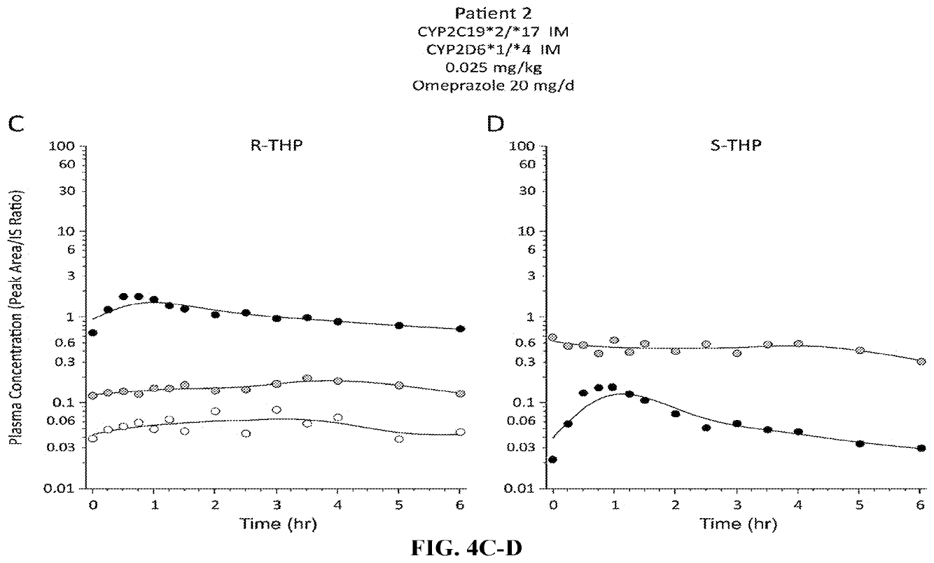

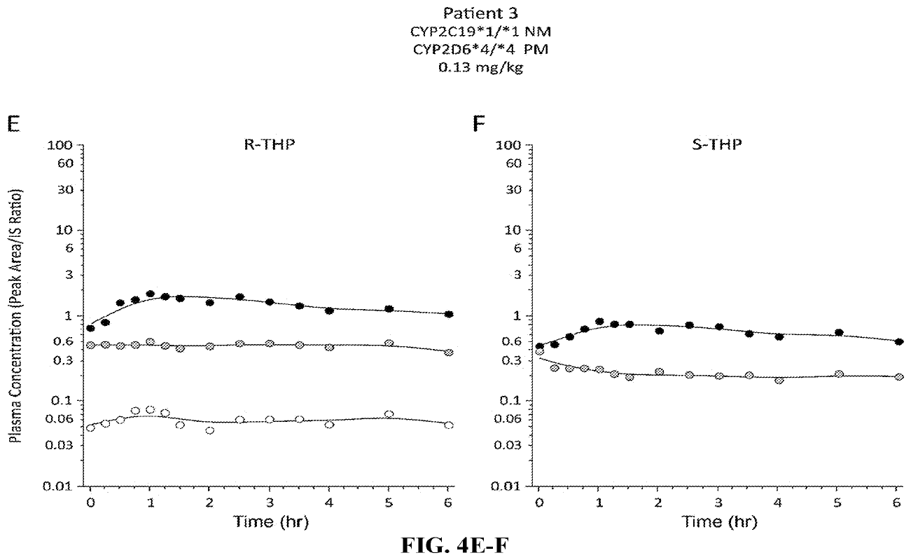

We investigated this by determining the impact of CYP2D6 and CYP2C19 metabolizer genotype-predicted phenotype on concentrations of S-trihexyphenidyl and R-trihexyphenidyl metabolites present in patient plasma. In addition to interindividual variability in CYP2C19 and CYP2D6 genotype, the results in FIG. 4, graphs A-F, demonstrate that metabolite patterns in three patients differed with respect to patient CYP2C19 and CYP2D6 genotypes, the dose of racemic trihexyphenidyl prescribed, and concurrent administration of an inhibitor of CYP2C19 activity. In each graph, the disposition of S-trihexyphenidyl or R-trihexyphenidyl is shown with the black circles, while the M1 metabolite concentrations are indicated by the gray circles, and M2 metabolite concentrations indicated by the white circles. Note that no CYP2D6-dependent S-THP-M2 concentrations were detected in patient 2, a CYP2D6 intermediate metabolizer in whom CYP2D6 activity is predicted to be reduced, or patient 3, a CYP2D6 poor metabolizer in whom activity is absent. Concentrations of the CYP3A4-dependent S-THP-M1 metabolite are present in all three patients and exceed S-trihexyphenidyl concentrations in patients 1 and 2. For R-THP, CYP2C19-dependent R-THP-M1 concentrations are highest relative to R-trihexyphenidyl plasma concentrations in patient 3 with the normal metabolizer CYP2C19*1/*1 genotype and lowest relative to R-trihexyphenidyl plasma concentrations in patient 1, who has an intermediate CYP2C19*1/*2 genotype and is receiving the highest dose of CYP2C19 inhibitor, esomeprazole 40 mg/day.

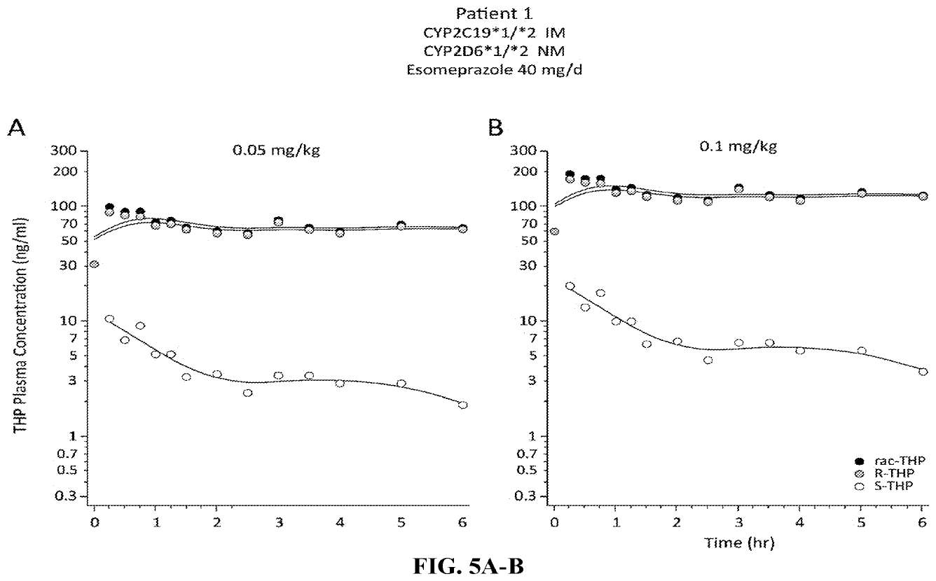

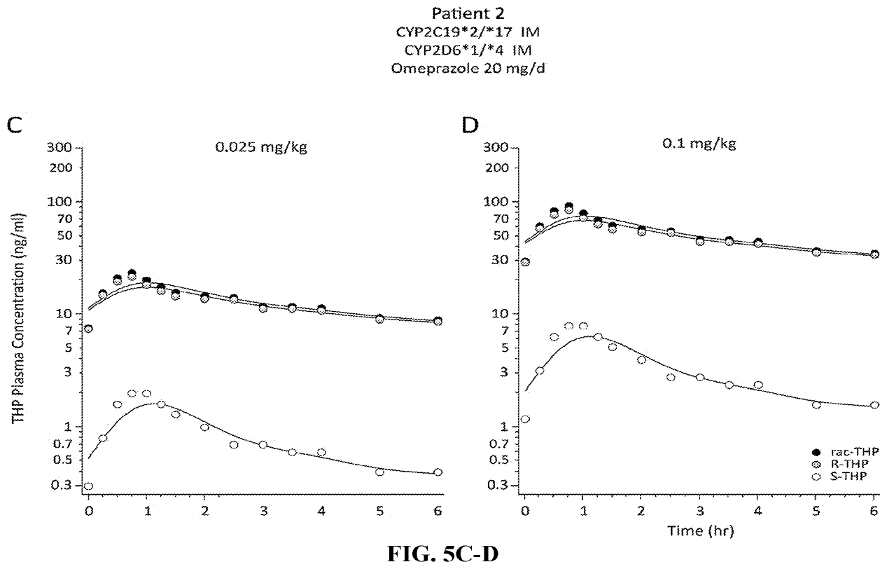

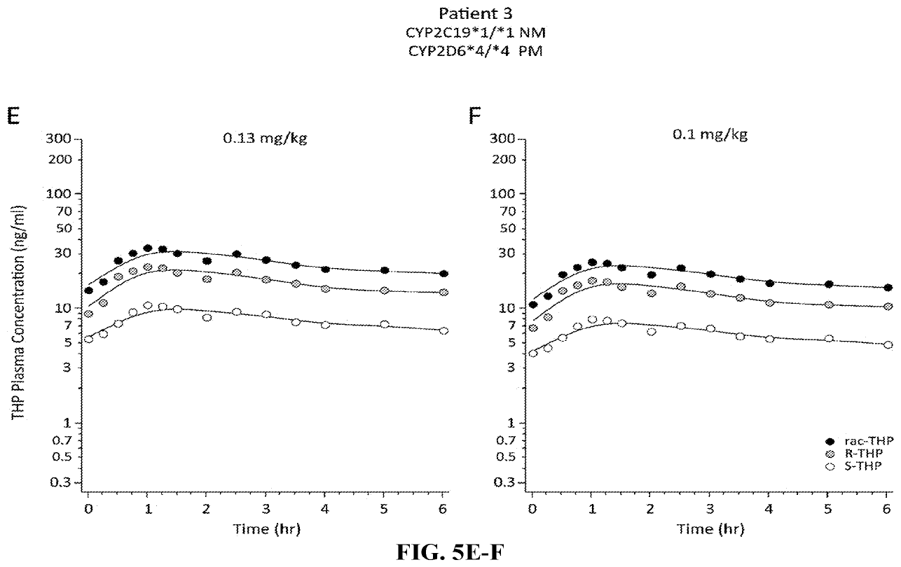

The consequences of interindividual variability in CYP2C19- and CYP2D6-dependent trihexyphenidyl metabolism described in FIG. 4 are presented in FIG. 5 for the same three patients. Steady state concentrations of racemic trihexyphenidyl (black circles) and for the two individual enantiomers, S-trihexyphenidyl (white circles) and R-trihexyphenidyl (gray circles), were measured over an entire 6-hour dosing interval. Each patient is unique with respect to CYP2C19 and CYP2D6 genotype, dose of racemic trihexyphenidyl prescribed, and concurrent administration of an inhibitor of CYP2C19 activity; each pair represents the results for a single patient, with the respective genotypes, racemic trihexyphenidyl doses and dose of inhibitor indicated above each column. In each pair, the lefthand graph represents the plasma concentrations for the actual dose prescribed and the righthand graph represents the concentration-time profiles corrected for a common dose of 0.1 mg/kg racemic trihexyphenidyl. In other words, assuming linear kinetics, righthand graphs indicate the concentrations that would be expected following a 0.1 mg/kg dose to each patient. The shapes of the curves are the same, but the curves are shifted upward two-fold and four-fold for patients 1 and 2, respectively. The curves for patient 3 are shifted lower since the actual dose was higher than 0.1 mg/kg. The similarity of the racemic (black) curves and the R-trihexyphenidyl (gray) disposition in patients 1 (CYP2D6 normal metabolizer; NM) and 2 (CYP2D6 intermediate metabolizer; IM) indicate that the majority of trihexyphenidyl present in the plasma of these patients is present as R-trihexyphenidyl. For patient 3 (CYP2D6 poor metabolizer; PM), S-trihexyphenidyl (white circles) concentrations represent approximately 32% of the total trihexyphenidyl present, compared to 5-6% for the other two patients. Expressed differently, in patient 3 S-trihexyphenidyl concentrations are about 50% of the R-trihexyphenidyl concentrations. In all patients, R-trihexyphenidyl concentrations are greater than S-trihexyphenidyl concentrations and tend to decline more slowly in patients 1 and 2 due to intermediate metabolizer CYP2C19 genotypes and the presence of inhibitors of CYP2C19 activity, esomeprazole and omeprazole, respectively.

These data indicate that THP is stereoselectively metabolized through hydroxylation of the cyclohexane ring where R-THP is metabolized primarily by CYP2C19 and S-THP is metabolized primarily by CYP2D6 in vitro. Collectively, these observations are consistent with formation of R-THP-M1 by CYP2C19 being the primary pathway involved in clearance of R-trihexyphenidyl (reduced enzyme activity due to genetic variation and concurrent administration of an inhibitor affect residual enzyme activity), and CYP2D6-dependent formation of S-THP-M2 influencing the reduced clearance, and thereby accumulation of higher concentrations, of S-trihexyphenidyl (potentially increasing chance of adverse drug reactions).

From these preliminary results, we propose that R-trihexyphenidyl would be superior to the racemic mixture as the R-enantiomer is more selective for M1 and M4 muscarinic receptors that are involved in movement disorders, including dystonia, and more specifically, Parkinson's, cerebral palsy, and Angelman syndrome. The S-enantiomer is not selective for muscarinic subtypes and may actually contribute to side effects due to its interactions with M1-M5 and activation of peripheral muscarinic receptors suggesting that it may be associated with side events, especially in CYP2D6 poor metabolizers. Administering R-THP as a single enantiomer may be safer and more effective. The data also demonstrates that the R- and S-enantiomers have distinct metabolic and clearance pathways (metabolized by different ADMERs); this observation may have important pharmacogenomic applications for individualization of drug therapy including by adjusting dosage recommendations based upon CYP2D6 and CYP2C19 metabolizer status of an individual.

1. A therapeutic composition comprising enantiomerically enriched R-trihexyphenidyl or a pharmaceutically acceptable salt thereof, dispersed in a pharmaceutically acceptable carrier.

2. The composition of claim 1, wherein said composition comprises an enantiomeric excess of at least about 75% of R-trihexyphenidyl.

3. The composition of claim 1, wherein said composition comprises an enantiomeric excess of at least about 85% of R-trihexyphenidyl.

4. The composition of claim 1, wherein said composition comprises an enantiomeric excess of at least about 95% of R-trihexyphenidyl.

5. The composition of claim 1, wherein said composition comprises an enantiomeric excess of at least about 99% of R-trihexyphenidyl.

6. The composition of claim 1, wherein said composition comprises less than 25% S-trihexyphenidyl.

7. The composition of claim 1, wherein said R-trihexyphenidyl is at least 95% enantiomerically pure.

8. The composition of claim 1, wherein said R-trihexyphenidyl is a pharmaceutically acceptable salt form selected from the group consisting of hydrochloride, hydrobromide, acetate, benzoate, carbonate, mesylate, and bitartrate.

9. A pharmaceutical dosage form comprising a therapeutically effective amount of the composition of claim 1.

10. The dosage form of claim 9, wherein said dosage form is a tablet, capsule, or oral solution.

11. The dosage form of claim 9, wherein said therapeutically effective amount is a dosage of from 3 mg per day to 30 mg per day.

12. A method of selectively targeting M1 and/or M4 muscarinic receptors in a subject in need thereof, the method comprising administering a therapeutically effective amount of enantiomerically enriched R-trihexyphenidyl or pharmaceutically acceptable salt thereof to the subject, wherein the R-trihexyphenidyl selectively binds to M1 and/or M4 in the subject.

13. The method of claim 12, orally administering a dosage form containing R-trihexyphenidyl wherein said dosage form comprises an enantiomeric excess of at least about 75% of R-trihexyphenidyl.

14. The method of claim 13, wherein dosage form contains purified R-trihexyphenidyl, and more preferably is substantially free of S-trihexyphenidyl enantiomer.

15. The method of claim 13, wherein said dosage form is a tablet, capsule, or oral solution.

16. The method of claim 13, the method comprising administering the R-trihexyphenidyl at a dosage of from 3 mg per day to 30 mg per day.

17. The method of claim 12, wherein said subject exhibits a reduction in the number, frequency, or severity of uncontrolled movements, spasms, or exhibits an improvement in measurements in gross or fine motor function tasks, after administration of said R-trihexyphenidyl.

18. Use of a medicament for selectively targeting M1 and/or M4 muscarinic receptors or treating dystonia in a subject in need thereof, said medicament comprising a therapeutic composition comprising enantiomerically enriched R-trihexyphenidyl or a pharmaceutically acceptable salt thereof according to any one of claims 1-8 or a pharmaceutical dosage form according to any one of claims 9-11.

19. An improved method for treating movement disorders in a subject in need thereof who is a poor CYP2D6 or CYP3A4/CYP3A5 metabolizer, the method comprising:
obtaining the CYP450 metabolic phenotype of the subject, and
administering a therapeutically effective amount of enantiomerically enriched R-trihexyphenidyl or a pharmaceutically acceptable salt thereof to the subject wherein the subject is a CYP2D6 or CYP3A4/CYP3A5 poor metabolizer.

20. An improved method for treating movement disorders in a subject in need thereof who is a CYP2C19 poor metabolizer, the method comprising:
obtaining the CYP450 metabolic phenotype of the subject, and
administering a therapeutically effective amount of enantiomerically enriched R-trihexyphenidyl or a pharmaceutically acceptable salt thereof to the subject wherein the subject is a CYP2C19 poor metabolizer, wherein said therapeutically effect amount is a low dose of said R-trihexyphenidyl as compared to a dose recommended in the clinical guidelines for trihexyphenidyl.

21. The method of claim 20, wherein said low dose is half the standard recommended dosage, preferably wherein said low dose is from 3 mg per day to 15 mg per day.

22. An improved method for treating movement disorders in a subject who is a poor CYP2D6 or CYP3A4/CYP3A5 metabolizer to minimize side effects, the method comprising:
obtaining the CYP450 metabolic phenotype of the subject, and
decreasing an initial dose of a racemic mixture of trihexyphenidyl or a pharmaceutically acceptable salt to less than 1 mg per day and administering to the subject wherein the subject is a CYP2D6 or CYP3A4/CYP3A5 poor metabolizer, or
increasing an initial dose of a racemic mixture of trihexyphenidyl or a pharmaceutically acceptable salt to more than 6 mg per day and administering to the subject wherein the subject is a CYP2D6 or CYP3A4/CYP3A5 normal or ultrarapid metabolizer,
wherein the initial dose is based upon an initial dose recommended in the clinical guidelines for trihexyphenidyl.

23. A method of administering an initial dose of a M1 and/or M4 muscarinic receptor inhibitor to a subject in need thereof, the initial dose based upon an initial dose recommended in the clinical guidelines for the inhibitor, the method comprising:
obtaining the subject's genotype for a panel of cytochrome P450 enzymes comprising at least CYP2D6 and/or CYP2C19 alleles, wherein the subject is assigned a metabolic phenotype selected from poor metabolizer, intermediate metabolizer, or ultrarapid metabolizer for each enzyme based upon the number of functional alleles for each cytochrome P450 gene;
administering to the patient an initial dose of the inhibitor, wherein said initial dose is:
(a) an initial dose that is half the initial dose recommended in the clinical guidelines if the metabolic phenotype is one or more of CYP2D6 poor metabolizer, CYP2D6 intermediate metabolizer, or CYP2C19 poor metabolizer; or
(b) an initial dose that is the same or higher than the initial dose recommended in the clinical guidelines if the metabolic phenotype is one or more of CYP2D6 ultrarapid metabolizer or CYP2C19 ultrarapid metabolizer; and
wherein the inhibitor is selected from the group consisting of a racemic mixture of trihexyphenidyl, enantiomerically enriched R-trihexyphenidyl, and pharmaceutically acceptable salts thereof.

24. A method of reducing side effects from an initial dose of a M1 and/or M4 muscarinic receptor inhibitor in a subject in need thereof, the initial dose based upon an initial dose recommended in clinical guidelines for the inhibitor, the method comprising:
obtaining the subject's genotype for a panel of cytochrome P450 comprising at least CYP2D6 alleles, wherein the subject is assigned a metabolic phenotype selected from poor metabolizer, intermediate metabolizer, or ultrarapid metabolizer for each enzyme based upon the number of functional alleles for CYP2D6;
administering to the patient an initial dose of the inhibitor, wherein said initial dose is:
(a) an initial dose that is half the initial dose recommended in the clinical guidelines if the metabolic phenotype is of a CYP2D6 poor metabolizer or CYP2D6 intermediate metabolizer; or
(b) an initial dose that is the same or higher than the initial dose recommended in the clinical guidelines if the metabolic phenotype is of a CYP2D6 ultrarapid metabolizer; and
wherein the inhibitor is a racemic mixture of trihexyphenidyl or a pharmaceutically acceptable salt thereof.

25. A method according to any one of claims 19-24, wherein said therapeutically effective amount or initial dose amount is on a per day basis, and wherein said trihexyphenidyl or a pharmaceutically acceptable salt thereof is administered, once, twice, or three times per day to reach said therapeutically effective amount or initial dose amount per day.
